(19) United States
(12) Patent Application Publication
(10) Pub. No.: US 2008/0103408 A1
(43) Pub. Date: May 1, 2008

(54) Continuous Intra-Abdominal Pressure Monitoring Urinary Catheter With Optional Core Temperature Sensor

(75) Inventors: Marshall T. Denton, Salt Lake City, UT (US); Timothy R. Wolfe, Salt Lake City, UT (US); Perry W. Croll, Salt Lake City, UT (US); Mark A. Christensen, Salt Lake City, UT (US)

Correspondence Address: TRASK BRITT, P.O. BOX 2550, SALT LAKE CITY, UT, 84110, US

(73) Assignee: Wolfe Tory Medical, Inc., Salt Lake City, UT (US)

(21) Appl. No.: 11/791,586

(22) PCT Filed: Nov. 21, 2005
(86) PCT No.: PCT/US05/42406

§ 371 Date: May 23, 2007

Related U.S. Application Data
(60) Provisional application No. 60/633,004, filed on Dec. 3, 2004.

(51) Int. Cl. A61B 5/03; A61B 5/01; A61M 27/00

(52) U.S. Cl. 600/549; 600/561; 600/581

(57) ABSTRACT

A device structured to generate an input signal to a monitor (201) to visually indicate a value for one or more physiological state variables measured by one or more transducers (203) associated with a medical patient. Preferred embodiments include a pressure transducer (195) placed in fluid communication with the patient's bladder (177) effective to infer intra-abdominal pressure P2. Embodiments may also include a temperature transducer (143) configured to measure the temperature of fluid in/near the bladder (177) to infer core body temperature. Certain embodiments of the invention may include a second pressure transducer (235) configured to measure arterial blood pressure. In the latter case, signals received from the two pressure transducers (177, 235) may be manipulated to produce a third signal corresponding to abdominal perfusion pressure, which can then be indicated on a numeric display device (233).

### PRIORITY CLAIM

This application claims the benefit under 35 U.S.C. 119(e) of the filing date of U.S. Provisional Patent Application Ser. No. 60/633,004, filed Dec. 3, 2004, for “CONTINUOUS INTRA-ABDOMINAL PRESSURE MONITORING URINARY CATHETER WITH CORE TEMPERATURE SENSOR.”

### TECHNICAL FIELD

The invention relates generally to plumbing devices that may include valves, conduits, temperature transducers, and pressure measurement devices. The invention relates particularly to apparatus configured as an assembly adapted to infer core body temperature and/or intra-abdominal pressure of a medical patient by measuring the temperature and hydraulic pressure of fluid associated with the patient's bladder.

### BACKGROUND

Elevated intra-abdominal pressure (IAP) leads to major changes in the body's physiology that, if undetected and untreated, can result in organ damage and patient death. When patients become critically ill, they may develop a capillary leak phenomenon that causes the tissues in their body to become edematous with extra fluid that seeps out of the capillaries. This process is called “3rd spacing” of fluid. It is very common in sepsis, burn, trauma and post-operative patients. One area of the body where 3rd spacing is especially prevalent is the abdominal cavity. Critically ill patients can have many liters of fluid leak into the intestinal wall, the intestinal mesentery, and the abdominal cavity (as free fluid sloshing around the intestines).

Fluid 3rd spacing in the abdominal cavity results in an increase in IAP. Normal IAP is 0 mm Hg to subatmospheric (less than 0). Once the pressure builds to 12-15 mm Hg, intra-abdominal hypertension (IAH) occurs. At this point, methods to improve intestinal perfusion should be started, such as: fluid loading to increase blood flow to gut, inotropic support to increase cardiac output, etc. As pressures increase above 20-25 mm Hg, the abdominal compartment syndrome (ACS) exists and major physiologic and organ system dysfunction result. Decompressive surgery (e.g. vertical midline abdominal incision) is often required to prevent irreversible organ damage and death. The exact pressure at which abdominal decompression should occur is dependent on a number of host factors including age, underlying co-morbidities and physiologic evidence of developing ACS.

Early detection of increasing abdominal pressure allows the clinician to intervene before irreversible organ damage occurs and may be life saving. The only reliable method for early detection of increasing IAP is to place a catheter within a space in the abdomen (peritoneal cavity, stomach, bladder, rectum) and measure the pressure. The most commonly used method is to monitor bladder pressure through an indwelling Foley catheter. To monitor bladder pressure, some clinicians are currently building their own devices out of many separate materials and inserting them into the Foley catheter.

Assessment of body temperature is essential in the clinical setting to ascertain baseline measures and to assess patients' response to, or the effectiveness of, treatments. Measurement of this vital sign is particularly important in critical care patients, whose thermostability may be challenged during recovery from surgery or as a result of inflammation, infection, or sepsis. Thermal instability, in turn, can induce hemodynamic or respiratory crises.

Although body temperature can be measured by using a variety of sites and devices, continuous measurement of core temperature, particularly on a long-term basis, has been problematic. Indwelling thermistor-tipped catheters or probes, such as those flow directed into the pulmonary artery, have been used mainly in intensive care units (ICUs), but only for selected patients who require hemodynamic monitoring. Esophageal probes have been used mainly in the operating room, but esophageal temperature is rarely monitored in critical care areas, and placement of the probe varies. Last, rectal probes have been used, mostly in the emergency department, for continuous monitoring of hypothermic or hyperthermic patients. The urinary bladder has become a more common and more widespread site for continuous monitoring of body temperature, particularly for patients who also require an indwelling catheter for urine drainage.

It is now well known in the medical field that bladder pressure correlates very closely to IAP. IAP is one physiological state variable that many health practitioners truly desire to know, but measured bladder pressure is used because it is a simple and relatively non-invasive way to obtain pressure readings closely approximating the true IAP. Bladder fluid temperature, as measured using instrumentation disposed inside a urinary catheter, is also deemed in the field as a sufficiently accurate approximation of core body temperature.

It would be an improvement in the art to provide a simple, rugged, and cost-effective apparatus operable to infer a patient's core body temperature and IAP. A further advance would provide an apparatus operable to collect such data on a substantially continuous basis. A still further advance would provide an apparatus capable of calculating and displaying a direct value effective to characterize one or more physiological state variables present in a medical patient, such as a difference in pressure (e.g. Abdominal Perfusion Pressure (APP)). It would be a further advance to provide an apparatus operable to display core body temperature in addition to a calculated physiological state variable.

### DISCLOSURE OF THE INVENTION

The present invention provides an apparatus that may be used for measuring bladder hydraulic pressure and/or bladder fluid temperature in a medical patient. Such pressure information may be collected on either a substantially continuous basis, or on an intermittent basis, as desired. Certain preferred embodiments structured according to principles of the instant invention may be configured to display a numerical value, or graphic, corresponding to one or more physiological states (such as IAP, or temperature) based upon one or more direct measurements. Certain other preferred embodiments may be configured to display a numerical value, or graphic, corresponding to one or more calculated physiological states (such as APP) based upon a plurality of measured input parameters.

An apparatus constructed according to certain principles of the invention to infer core body temperature and intra-abdominal pressure includes a urinary catheter having a distal end adapted for insertion into a patient's bladder. The catheter includes at least first and second lumens, although catheters having three or more lumens are also workable. The first lumen provides fluid communication between balloon inflation structure associated with a proximal end of the catheter and an inflatable balloon associated with a distal end of the catheter. The second lumen is configured to provide fluid communication between drain connection structure associated with the proximal end of the catheter and at least one draining port disposed distal to the balloon. Some operable catheters may includes a third lumen configured to provide fluid communication between infusion fluid connection structure associated with the proximal end of the catheter and at least one infusion aperture disposed distal to the balloon.

In any case, the catheter includes infusion fluid connection structure adapted to receive infusion fluid from a fluid source. Infusion fluid connection structure may be incorporated into structure of a catheter, or may include one or more separate components. One operable such connection structure includes a branched conduit, with a first branch including structure adapted to form a fluid resistant connection permitting communication between the infusion aperture and an infusion fluid source, and with a second branch carrying first seal structure adapted to form a fluid resistant seal with cooperating second seal structure associated with a transducer when that transducer is disposed at an installed position with respect to the second branch. In certain cases, infusion fluid connection structure can include a branched conduit having a first branch adapted to permit fluid communication between the catheter's draining port and an infusion fluid source, and with a second branch of said branched conduit being adapted to permit fluid flow between the draining port and a drain exit to a container.

A temperature transducer may be installed at a position effective to measure temperature corresponding to the temperature of fluid inside the bladder. Desirably, a length portion of the temperature transducer is installed inside the third lumen. In such case, length portion is desirably structured in harmony with a cross-section of the third lumen to permit flow of infusion fluid along an axis of the length portion for discharge of infusion fluid through the infusion aperture at a sufficient flow rate to permit substantially continuous monitoring of bladder pressure. However, a temperature transducer can alternatively be installed inside the second lumen, even if the catheter includes a third lumen. Furthermore, pressure measurements may alternatively be made on an intermittent basis, if desired.

A first pressure transducer is placed in fluid communication with the bladder to measure pressure of fluid inside the bladder, and to generate a corresponding first output signal. A second pressure transducer can be disposed to measure pressure corresponding to the blood pressure inside the patient, and to generate a corresponding second output signal. Certina embodiments include a processing unit adapted to receive the first and second output signals for manipulation, and to generate a resulting third output signal corresponding to a calculated physiological state variable. Desirably a display device is arranged to cause a visual display responsive to the third output signal. In a currently preferred embodiment, the first output signal correlates with bladder pressure in the patient; the second output signal correlates with arterial pressure in the patient; and the visual display correlates with Abdominal Perfusion Pressure.

The invention can be embodied as an apparatus adapted for monitoring a calculated physiological state variable of a medical patient based upon a plurality of transducer inputs. Such an apparatus includes a catheter configured and arranged to provide fluid communication with the patient's bladder. In such case, a first transducer is arranged in harmony with the catheter to measure a first physiological state variable inside the bladder and to produce a first output signal correlating with that first state variable. A second transducer is disposed at a different location associated with the patient to measure a second physiological state variable and to produce a second output signal correlating with the second state variable. Structure is provided to manipulate the first and second output signals to produce the calculated state variable and produce a third output corresponding to the calculated physiological state variable for input to a visible display device.

One preferred apparatus includes a first transducer to generate a first output signal correlating with bladder pressure in the patient. Such apparatus also includes a second transducer to generate a second output signal correlating with that patient's arterial blood pressure. Therefore, the resulting calculated state variable correlates with Abdominal Perfusion Pressure. Some embodiments of the invention may further include a third transducer arranged to measure a fourth physiological state variable inside the bladder and produce a corresponding output signal correlating with temperature of fluid in the bladder.

These features, advantages, and alternative aspects of the present invention will be apparent to those skilled in the art from a consideration of the following detailed description taken in combination with the accompanying drawings.

### BRIEF DESCRIPTION OF THE DRAWINGS

In the drawings, which illustrate what is currently considered to be the best mode for carrying out the invention:

FIG. 1 is a plan assembly view of a monitoring device constructed according to principles of the invention;

FIG. 2 is a cross-section view taken through section 2-2 in FIG. 1, and looking in the direction of the arrows;

FIG. 3 is an illustration of one workable plumbing arrangement for practice of the invention;

FIG. 4 is an illustration of a second workable plumbing arrangement for practice of the invention; and

FIG. 5 is an illustration of a patient connected to an assembly constructed according to certain principles of the invention.

### MODES FOR CARRYING OUT THE INVENTION

Reference will now be made to the drawings in which the various elements of the invention will be given numerical designations and in which the invention will be discussed so as to enable one skilled in the art to make and use the invention. It is to be understood that the following description is only exemplary of the principles of the present invention, and should not be viewed as narrowing the claims which follow.

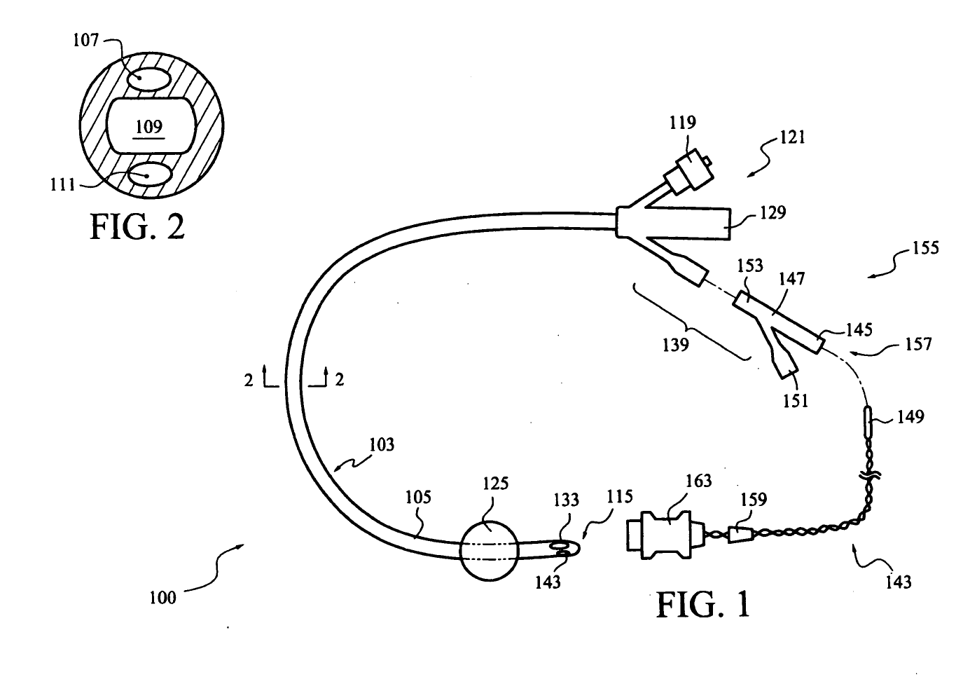

With reference to FIG. 1, a currently preferred monitoring device constructed according to certain principles of the invention is indicated generally at 100. Monitoring device 100 is typically included as a portion of an assembly structured to permit a clinical worker to monitor one or more physiological state variable in a medical patient. Monitoring can be accomplished on a discrete, or continuous basis with respect to time, depending upon the configuration of the monitoring assembly. Device 100 can be used as a portion of an apparatus adapted to monitor physiological state variables that nonexclusively include core body temperature, and pressure, such as abdominal pressure.

Illustrated monitoring device 100 includes a urinary catheter 103, which includes an elongated tubular body member, generally indicated at 105, having at least three lumens. With reference to FIG. 2, catheter 103 includes first lumen 107, second lumen 109, and third lumen 111. It should be noted that embodiments structured according to principles of the instant invention may be constructed having two lumens, or more than three lumens. In any case, the distal end 115 of the catheter 103 is desirably blunted to facilitate placement of the distal end 115 inside a patient. One operable catheter for construction of certain embodiments of the invention is a Foley catheter commercially available from C.R. Bard, Inc., of Covington Ga. under the part No. 73018L.

In general, one lumen (e.g. lumen 107 in FIG. 2) of the urinary or Foley catheter 103 is adapted to permit communication between inflation structure, generally indicated at 119, associated with a proximal end 121 of catheter 103 and an inflatable balloon 125 disposed near distal end 115 of the catheter 103. When inflated, balloon 125 serves as a restraint to resist inadvertent removal of the catheter 103 from placement inside the patient.

A second lumen (e.g. lumen 109) provides fluid communication between drain connection structure 129 associated with the proximal end 121 of the catheter 103 and at least one draining port 133 disposed distally of the balloon 125. During use, urine and other fluids are permitted to drain from the patient's bladder through the second lumen on either a continuous, or intermittent, basis.

If present, a third lumen (e.g. lumen 111) may provide fluid communication between fluid connection structure, e.g. generally indicated at 139, associated with proximal end 121 of the catheter 103 and at least one infusion aperture 143 disposed distal to the balloon 125. In one preferred arrangement, infusion fluid may be pumped into the patient's bladder through the third lumen.

As illustrated in FIG. 1, a temperature transducer, generally indicated at 143, may be included in certain embodiments of the invention to permit measuring the temperature inside the abdomen area when a catheter 103 is installed into a patient. A workable temperature transducer is commercially available from G.E. Thermometrics, of Edison N.J., under the part No. A329. A sufficient length of the temperature transducer 143 can be conveniently installed through a first branch 145 of a branched conduit 147 to place the thermocouple 149, or other temperature measuring element, at an operable and effective location for registration at a desired position inside the patient's body effective to record temperature correlating with core body temperature. Desirably, the thermocouple 149 is placed inside the third lumen. Placement of thermocouple 149 inside the third lumen is effective to resist occlusion of the urine drain path. Furthermore, infusion fluid can flush the third lumen and help maintain its cleanliness. Infusion fluid can be placed into fluid communication with, and urged to flow through, second branch 151.

A distal end 153 of the branched conduit 147 can be joined to structure at a proximal end of the urinary catheter in any operable and well known way, including luer-type connections, or simple friction-fit joint structure. In an alternative construction (not illustrated), structure equivalent to the branched conduit can be formed as a portion of the urinary catheter 103. A catheter constructed substantially according to the alternative suggested construction is commercially available from Smiths Medical under part No. FC400-16. However, the FC400-16 catheter requires modification to place the lumen in which the temperature sensor is desirably installed into open fluid communication with an infusion aperture disposed at the distal end of such catheter.

A fluid resistant seal arrangement, generally indicated at 155, is desirably provided between cooperating first seal structure 157 and second seal structure 159 to resist fluid leaks through the first branch 145. As illustrated, the first branch 145 carries a first seal structure 157 cooperatively shaped to engage the second seal structure 159, carried by the temperature transducer 143, when the temperature transducer 143 is installed into the branched conduit 147. The illustrated second seal structure 159 forms a plug fit connection inside the first seal structure 157.

It should be noted that the second seal structure 159 can be disposed at any desired position along a length of the temperature transducer 143. As illustrated, the second seal structure 159 is spaced apart from an electrical connector 163. However, it is within contemplation alternatively for the seal structure to be associated with a connector 163, e.g. to help maintain the connector 163 in a fixed position to facilitate making an intermediate connection to a monitor or recording device, and/or to protect the transducer wires from bending damage. In any case, it is desirable to form an assembly that places the connector 163 at a location unlikely to cause discomfort to the patient.

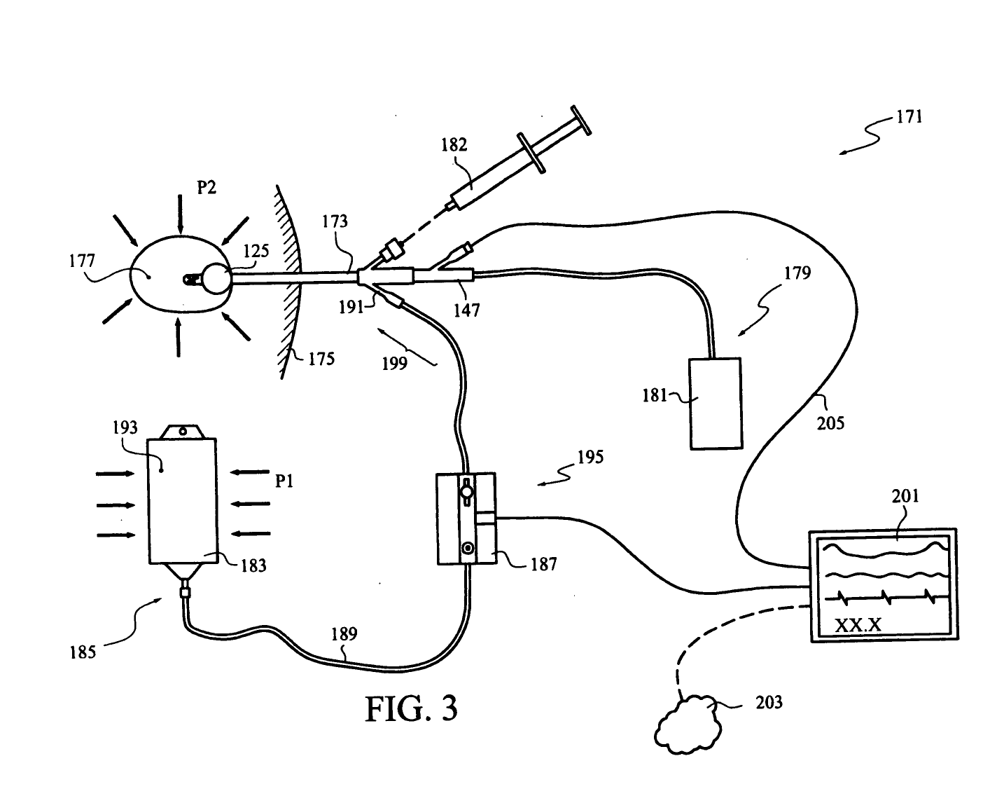

FIG. 3 illustrates a first assembly, generally indicated at 171, structured according to certain principles of the invention. A 3-way Foley catheter 173 is installed in a patient 175 and connected to continuously drain urine and fluids from the patient's bladder 177 through a drain exit 179 into a container 181. Syringe 182 can be used in a known manner to inflate the balloon 125 subsequent to installation of catheter 173 into the patient 175. A saline bag 183 may be tapped (e.g. with a spike connector 185), and connected in fluid communication, through a low-flow flush valve 187 disposed along fluid conduit 189, with the infusion line 191 of the Foley catheter 173. The saline bag 183 may then be pressurized (as indicated at P1), in a known structure to about 300 mm Hg, or simply suspended for gravity action on the infusion fluid 193. The low flow flush valve 187 desirably is effective to isolate the pressure P1 imposed on infusion fluid 193 in the bag 183 from a pressure transducer.

Sometimes, a pressure transducer, generally indicated at 195, is integrated into the low-flow flush valve 187 for convenience of assembly of the assembly 171. In such case, pressure transducer 195 is disposed downstream of the flow-control element 187. The impedance of the fluid conduit section 199 between the pressure transducer and bladder is sufficiently low, compared to the flow rate permitted by a preferred flow control device 187, that the pressure downstream of the low flow device 187 is substantially governed by the pressure (indicated at P2), in the bladder 177 (and patient abdomen). Therefore, pressure measured in infusion fluid downstream of the flow-control device 187 correlate to the bladder pressure in the patient 175.

One operable flush valve includes a valve commercially available from Edwards Lifesciences, under the part No. PX600F. Such flush valve typically permits a fluid flow from the described pressurized infusion fluid source 183 of about 3 ml/hr, and includes a stopcock and a pressure transducer 195 in an integrated assembly. An electrical signal from the pressure transducer 195 may be displayed on a monitor 201. The pressure in the patient's bladder (which corresponds to the abdominal pressure of that patient), can therefore be monitored continuously in the arrangement illustrated in FIG. 3.

Although it is counter-intuitive in an open-flow system, such as illustrated in FIG. 3, it has been determined that the arrangement illustrated in FIG. 3 is effectively adapted to continuously measure a pressure corresponding substantially to the patient's abdominal pressure, because structure associated with the bladder's wall and the infusion aperture cooperate to form a discharge-regulating valve. So long as the urine drain tube is free to drain fluids (e.g. the drain tube is not occluded by a closed valve), the draining port(s) remove substantially all urine and fluid from the patient's bladder. Therefore, the bladder is maintained in a substantially empty condition, and is collapsed onto the distal end of the catheter under effect of the intra-abdominal pressure. An occlusion is imposed over the infusion port, either by the bladder wall itself, or by mucus-like fluids associated with the wall. The continuous flow of infusion fluid must overcome the pressure imposed as an occlusion over the infusion orifice (by intra-abdominal pressure interacting with structure associated with the bladder), and therefore the infusion fluid remains at all times substantially at a pressure closely corresponding to the pressure inside the abdomen.

The discharge-regulating valve is believed to be effective because of the combination of: the infusion fluid flow rate is very low; the intra-abdominal pressure is fairly small (on the order of between 0-50 mm Hg); the bladder wall is flaccid, membrane-like, and conformable, and the intra-abdominal pressure is exerted upon the bladder in 3-D to press the bladder wall into engagement with the distal portion of the catheter effective to resist fluid flow through the infusion aperture. That being said, it is recognized that formation of the discharge-regulating valve is not necessarily guaranteed, e.g. due to potential misalignment of the relevant structures, or during sudden fluid draining episodes.

With continued reference to FIG. 3, it is sometimes desirable to optionally include provision to display the output obtained from one or more transducers 203. As one nonlimiting example, it is sometimes desirable to include a temperature transducer arranged to measure temperature of fluids in, or near, the bladder 177 to additionally monitor core body temperature along with IAP. As another nonlimiting example, data obtained from a plurality of pressure transducers may be manipulated to generate a resulting product value indicative of a physiological state variable that cannot easily be measured directly, such as APP.

As illustrated in FIG. 3, a temperature transducer lead wire 205 from an optional temperature transducer is connected to a monitor 201. In certain instances, monitor 201 can be configured to display a plurality of measured and/or calculated values, including pressure and temperature. In other instances, a plurality of monitor display devices may be incorporated to provide the desired visual display. Some physiological state variables, e.g. heart rate or certain variables that are measured continuously with respect to time, may best be displayed in a graphic form, such as a line plot. Other physiological state variables, such as APP, may be better displayed in discrete numeric form. Therefore, one or more display devices may be combined to form a structure equivalent to the illustrated monitor 201.

Note that FIG. 3 illustrates the branched conduit 147 being placed into fluid communication with a drain conduit through the catheter 173, rather than the infusion port as illustrated in FIG. 1. The illustrations demonstrate that various changes can be made to the plumbing arrangement of the various components, without departing from the spirit and essential characteristics of the instant invention. It should be noted that the arrangement illustrated in FIG. 3 is less preferred, because the temperature transducer, having a length portion disposed in the drain conduit (e.g. 109 in FIG. 2) of catheter 173, is bathed in a discharge stream from the patient's bladder 177, rather than being flushed clean by infusion fluid. However, the arrangement illustrated in FIG. 3 is workable, and also can provide the advantage of disposing the transducer in a bore of commercially available catheters having a larger cross-section size.

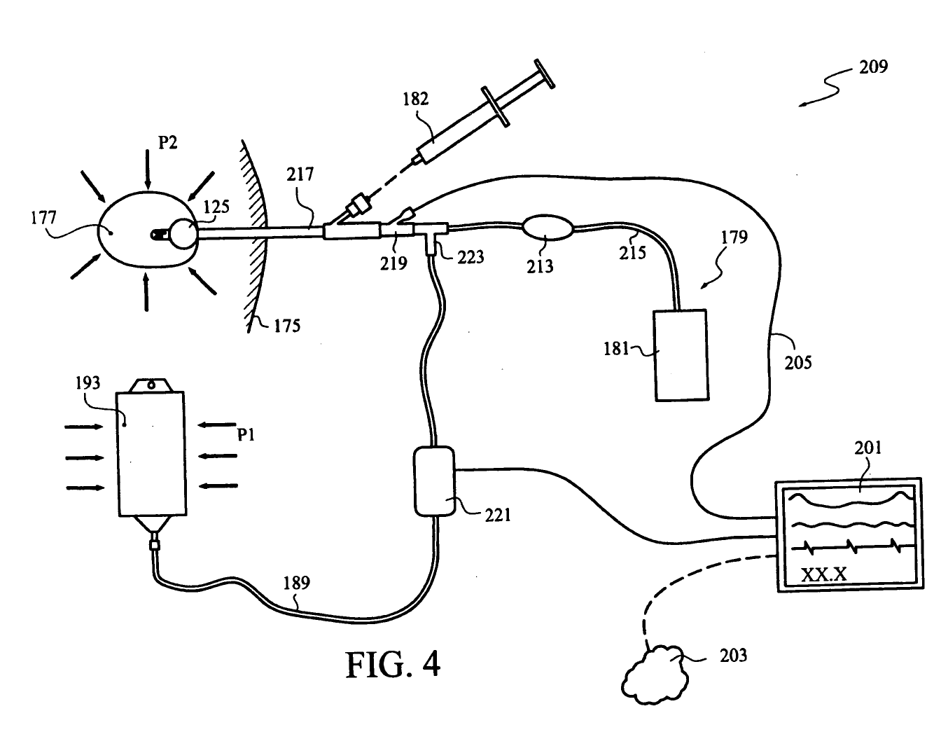

IAP can be measured at intermittent instances spaced apart in time, or continuously. Intermittent IAP measurements may be conveniently made in and alternate plumbing arrangement, such as is generally indicated at 209 in FIG. 4, and which includes connecting a urine drain-occluding valve 213 in circuit with the urine drain conduit 215. To collect intermittent IAP data, the drain-occluding valve 213 can be placed into a drain occluding configuration while a proscribed volume of infusion fluid 193 is introduced through the urinary catheter 217 into the patient's bladder. The fluid pressure in the bladder is measured, and then the urine drain is then returned to a non-occluded, or fluid draining configuration until the next measurement instance. Naturally, the intermittent procedure is less desirable because it typically requires time and attention of personnel, and may introduce a time delay between a significant IAP change and a measurement instance. In general, monitor structure 201 is adapted to receive input from two or more transducers 203.

Note that FIG. 4 illustrates use of a two-way urinary catheter 217 and another alternative plumbing arrangement within the ambit of the instant invention. An optional temperature transducer, if included, can be connected to a monitor 201 by way of lead wire 205. The temperature transducer can be introduced into catheter 217 through a branched conduit 219, similar to the arrangements described with reference to FIGS. 1 and 3. Also note that a combination flow control valve arrangement 221 is illustrated, which includes an integrated pressure transducer along with valve structure. Of course, structure that is hydraulically equivalent to components including branched conduit 219, integrated device 221, or fluid connector 223 may be provided, and the locations of respective components can be shuffled or reordered without departing from the spirit and essential characteristics of the invention.

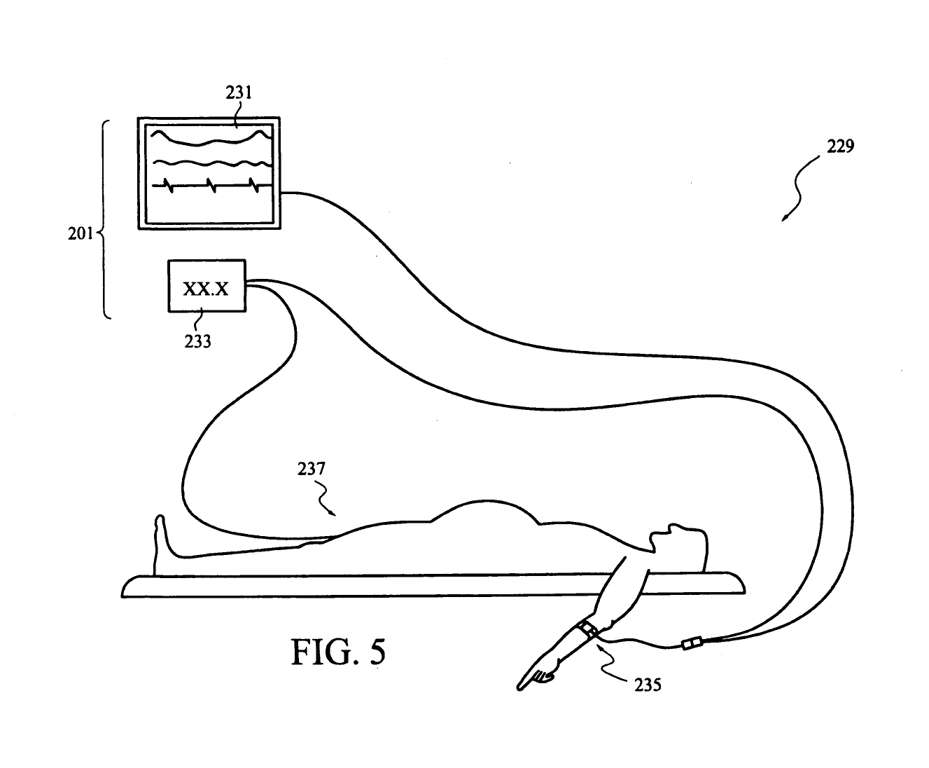

FIG. 5 illustrates an arrangement of components, generally indicated at 229, operable to display the patient's Abdominal Perfusion Pressure (APP). The APP is obtained by subtracting the patient's Mean Intra-Abdominal Pressure from the patient's Mean Arterial Pressure. Such calculated number is more effective to indicate the true state of the patient's risk of Abdominal Compartment Syndrome. A first patient having high arterial pressure may be able to tolerate an intra-abdominal pressure of an elevated amount. A second patient having low arterial pressure may be at significant risk from an intra-abdominal pressure of such elevated amount. In such case, the calculated APP number for the first patient would indicate a higher (safer) number, compared to the second patient.

The arrangement 229 illustrated in FIG. 5 includes a monitor structure 221 operable to display information corresponding to data received from a plurality of transducers 203. Monitor 221 includes a graphic display 231 and a numeric display 233. Graphic display 231 is representative of display devices currently commonly present in an intensive care unit, and may indicate one or more of a patient's heart rate, arterial blood pressure, CVP, specific oxygen uptake, and core body temperature, among other physiological state variables. Numeric display 233 may be used to indicate discrete numeric values, such as values corresponding to physiological state variables that may be intermittently acquired.

As illustrated in FIG. 5, numeric display 233 is adapted to receive inputs from two or more transducers. Each of such transducers are adapted to generate a signal corresponding to a physiological state variable. As one non-limiting example, FIG. 5 illustrates numeric display 233 receiving inputs from both of an ubiquitous arterial line pressure transducer 235, and an intra-abdominal pressure transducer 237. A first signal received from the arterial line pressure transducer and a second signal received from the intra-abdominal pressure transducer are manipulated by processing structure associated with display 233 to produce a third signal corresponding to a third physiological state variable, namely APP.

A workable intra-abdominal pressure transducer 237 can be embodied in various forms, such as the integrated device 187 illustrated in FIG. 3, or as a discrete component. The numeric monitor 233 typically includes hard-wired circuitry, and/or a processor, effective to make the calculation and is typically structured to display the desired APP number as a discrete numeric value. When the APP number drops below a certain threshold amount, such as perhaps 59 mm Hg, the surgeon knows that the patient must undergo an immediate surgical procedure.

What is claimed is:

1. An apparatus adapted for monitoring temperature and pressure corresponding to the state of fluid contained inside the bladder of a medical patient to infer core body temperature and intra-abdominal pressure, the apparatus comprising:
a urinary catheter having a distal end adapted for insertion into the bladder of a medical patient;
a first lumen associated with said catheter to provide fluid communication between balloon inflation structure associated with a proximal end of said catheter and an inflatable balloon associated with said distal end of said catheter;
a second lumen associated with said catheter to provide fluid communication between drain connection structure associated with said proximal end of said catheter and at least one draining port disposed distal to said balloon;
a temperature transducer adapted for disposition with respect to said catheter at a position effective to measure temperature corresponding to the temperature of fluid inside said bladder; and
a first pressure transducer disposed in fluid communication with said bladder effective to measure pressure corresponding to the pressure of fluid inside said bladder, and to generate a corresponding first output signal.

2. The apparatus according to claim 1, further comprising:
a third lumen associated with said catheter to provide fluid communication between infusion fluid connection structure associated with said proximal end of said catheter and at least one infusion aperture disposed distal to said balloon.

3. The apparatus according to claim 2, wherein:
said temperature transducer comprises a length portion disposed inside said third lumen.

4. The apparatus according to claim 3, wherein:
said length portion being structured in harmony with a cross-section of said third lumen to permit flow of infusion fluid along an axis of said length portion for discharge of said infusion fluid through said infusion aperture at a sufficient rate to permit substantially continuous monitoring of bladder pressure.

5. The apparatus according to claim 3, wherein:
said infusion fluid connection structure comprises a branched conduit, with a first branch of said branched conduit including structure adapted to form a fluid resistant connection permitting communication between said infusion aperture and an infusion fluid source, and with a second branch of said conduit carrying first seal structure adapted to form a fluid resistant seal with cooperating second seal structure associated with said temperature transducer when said temperature transducer is disposed at an installed position with respect to said second branch.

6. The apparatus according to claim 1, wherein:
said temperature transducer comprises a length portion disposed inside said second lumen.

7. The apparatus according to claim 2, wherein:
said temperature transducer comprises a length portion disposed inside said second lumen.

8. The apparatus according to claim 6, wherein:
said infusion fluid connection structure comprises a branched conduit, with a first branch of said branched conduit including structure adapted to permit fluid communication between said draining port and an infusion fluid source, and with a second branch of said branched conduit being adapted to permit fluid flow between said draining port and a drain exit.

9. The apparatus according to claim 1, further comprising:
a second pressure transducer disposed to measure pressure corresponding to the blood pressure inside said patient, and to generate a corresponding second output signal;
a processing unit adapted to receive said first output signal and said second output signal for manipulation, and to generate a resulting third output signal; and
a display device operable to cause a visual display responsive to said third output signal; wherein:
said first output signal correlates with bladder pressure in said patient;
said second output signal correlates with arterial pressure in said patient; and
said visual display correlates with Abdominal Perfusion Pressure.

10. An apparatus adapted for monitoring at least a first physiological state variable of a medical patient, the apparatus comprising:
a catheter configured and arranged to provide fluid communication with the bladder of said patient;
a first transducer arranged in harmony with said catheter effective to measure a second physiological state variable inside said bladder, said first transducer producing a first output signal correlating with said second variable;
a second transducer disposed at a different location associated with said patient effective to measure a third physiological state variable, said second transducer producing a second output signal correlating with said third variable; and
a display device associated with said first transducer and said second transducer effective to output a visual display corresponding to said first physiological state variable; wherein:
a generated display signal to cause said visual display of said first variable is obtained by manipulation of said first output signal and said second output signal.

11. The apparatus according to claim 10, wherein:
said first output signal correlates with bladder pressure in said patient;
said second output signal correlates with arterial pressure in said patient; and
said first variable correlates with Abdominal Perfusion Pressure.

12. The apparatus according to claim 10, further comprising:
a third transducer arranged in harmony with said catheter effective to measure a fourth physiological state variable inside said bladder, said third transducer producing a third output signal correlating with temperature of fluid in said bladder.
